(19) United States
(12) Patent Application Publication
(10) Pub. No.: US 2024/0296959 A1
(43) Pub. Date: Sep. 5, 2024

(54) ASSESSMENT OF RISK FOR MAJOR ADVERSE CARDIAC EVENT

(71) Applicant: Beckman Coulter, Inc., Brea, CA (US)

(72) Inventors: Sean M. ROBERTS, Brea, CA (US); Girish Simon, Brea, CA (US)

(21) Appl. No.: 18/661,180

(22) Filed: May 10, 2024

Related U.S. Application Data
(63) Continuation of application No. PCT/US2022/079192, filed on Nov. 3, 2022.
(60) Provisional application No. 63/263,906, filed on Nov. 11, 2021.
(60) Provisional application No. 63/263,908, filed on Nov. 11, 2021.
(60) Provisional application No. 63/263,911, filed on Nov. 11, 2021.

(51) Int. Cl. G16H 50/30; G16H 10/60

(52) U.S. Cl. CPC G16H 50/30; G16H 10/60

(57) ABSTRACT

A computer-implemented method of determining a major adverse cardiovascular event, risk of MACE, in a patient, is provided. The method comprises receiving, with a computing device, subject value data for the patient, the subject value data including (i) at least one troponin value, (ii) at least one demographic value, and (iii) at least one of a value for prior history of cardiac disease, a value of prior history of renal disease, an erythrocyte mean corpuscular hemoglobin value and an electrolyte value. Further, the method comprises evaluating, with the computing device, the received subject value data of the patient based on a reference dataset indicative of reference subject values associated with one or more reference patients, wherein the reference dataset is indicative of reference subject values including (i) at least one troponin value, (ii) at least one demographic value, and (iii) a value for prior history of cardiac disease. Further, the risk of MACE is determined based on the evaluation.

### CROSS-REFERENCE TO RELATED APPLICATIONS

This application claims the benefit of U.S. Provisional Application Nos. 63/263,906; 63/263,908; and 63/263,911 all of which were filed on Nov. 11, 2021 and all of which are incorporated by reference as if fully set forth herein.

### TECHNICAL FIELD

The present disclosure generally relates to the field of clinical decision support. In particular, the present disclosure relates to one or more computer-implemented methods of determining risk for a major adverse cardiovascular event (referred to herein as “MACE”) in a patient. Exemplary patients include, for example a patient with prior history of cardiac disease, a patient without prior history of cardiac disease, a patient with prior history of renal or kidney disease, and/or a patient with current symptoms of renal disease or kidney disease. Further, the present disclosure relates to a computer program for carrying out steps of said method, to a computer-readable medium, for example a non-transitory computer-readable medium, storing such computer program, and to a computing device configured to perform steps of said method.

### BACKGROUND

An increasing number of patients are assessed in hospitals for a health-relevant cardiac event and/or for a likelihood of a future major adverse cardiovascular event (MACE) developing in a patient within a certain period of time after an assessment at the hospital. Generally, patients can be diagnosed or assessed at a hospital for an emergent health-relevant cardiac event are suspected of having an acute coronary syndrome (also referred to herein as “ACS”), a range of serious conditions involving the heart, for example, myocardial injury, ischemic cardiovascular events, heart failure, myocardial infarction, myocardial or pericardial infection, or stroke. The risk of MACE, being the risk of a potential adverse future outcome, is a common measure of the safety profile for discharging a patient. That is, the likelihood of a future negative event is often used to inform the decision to discharge a patients or admit them for further examination. In some instances, the risk of MACE is evaluated after the evaluation for or diagnosis of a health-relevant cardiac event. Accordingly, a patient may be evaluated for a health-relevant cardiac event at a hospital or emergency department, and the risk or likelihood of MACE for the patient can additionally be evaluated or prognosed. In this context, the risk or likelihood for MACE can be considered as a prognostic risk or likelihood of an adverse or negative outcome of a health-relevant cardiac event potentially occurring or developing in a patient. In some aspects, the risk of MACE is determined for a certain time period. For example, the risk of MACE may be determined for a certain period of time after the assessment of the cardiac event at the hospital, such as within 7 days of the assessment of the cardiac event, within 30 days of the assessment of the cardiac event at the hospital, within 60 days of the assessment of the cardiac event at the hospital, or within 90 days of the assessment of the cardiac event at the hospital.

Patients assessed for a health-relevant cardiac event such as ACS at a hospital or emergency department typically report chest pain, shortness of breath, and often additional symptoms suggestive of cardiac involvement. The standard test for diagnosing a health-relevant cardiac event, among suspect ACS patients at hospitals or emergency departments includes a troponin (Tn) serum assay and ECG/EKG, for example used in conjunction with clinical data, to determine if a patient is suffering cardiac injury. Moreover, high-sensitivity troponin testing has been shown to aid with the risk stratification of a patient assessed for ACS, including risk for MACE. These high-sensitivity troponin assays can detect lower levels of troponin in the blood with analytical sensitivities up to 100 times greater than conventional troponin assays. Use of high-sensitivity troponin assays can enable the detection of small changes in troponin levels or values and can help identify patients exhibiting cardiac injury (also referred to as “cardiac patients”) and patients unlikely to be exhibiting cardiac injury (also referred to as “non-cardiac patients”), for example to help triage patients more accurately and rapidly. Although the troponin level can be an effective indicator for the risk for MACE in some patients, it may be challenging to accurately rule in or rule out a patient as having a high risk for MACE based on the troponin level or value alone.

### SUMMARY

It may therefore be desirable to provide for an improved method and device for determining a risk, probability, and/or likelihood of MACE in a patient, for example allowing to efficiently, reliably, and accurately rule in or rule out a patient as a cardiac patient and/or assessing the likelihood of a patient to experience a negative medical outcome (i.e. MACE), e.g. within the specified time period.

This is achieved by the subject matter of the independent claims. Exemplary embodiments are incorporated in the dependent claims and the following description.

Aspects of the present disclosure relate to one or more computer-implemented methods of determining and/or assessing a risk of MACE (e.g., 30 day MACE) in a patient, for example, in a patient with prior history of cardiac disease, without prior history of cardiac disease, in a patient with prior history renal disease, in a patient with current symptoms of renal disease, and/or in a so-called indeterminate or indeterminant patient (as further described herein below), to a computer program for carrying out steps of one or more of said methods, to a computer-readable medium, for example a non-transitory computer-readable medium, storing such computer program, and to a computing device configured to perform steps of one or more of said methods. Any disclosure presented herein above and herein below with reference to one aspect of the present disclosure equally applies to any other aspect of the present disclosure.

An aspect of the present disclosure relates to a computer-implemented method of determining and/or assessing a risk of MACE in a patient. A further aspect of the present disclosure relates to a computer-implemented method of determining and/or assessing a risk of MACE in a patient with prior history of cardiac disease. Yet another aspect of the present disclosure relates to a computer-implemented method of determining and/or assessing a risk of MACE in a patient with prior history of renal disease. Yet another aspect of the present disclosure relates to a computer-implemented method of determining and/or assessing a risk of MACE in an indeterminate patient, as will be further discussed herein below. Any one or more of these methods may, alternatively or additionally, refer to a computer-implemented method of prognosing MACE in the patient. Further, it is noted that determining and/or assessing the risk of MACE in a patient may refer to determining the risk of MACE in the patient within a certain period of time following a physician assessment of a health-relevant cardiac information. Since the following disclosure equally applies to any one or more of the aforementioned methods, it may be referred to “the method” in the following for simplicity. Any reference to a singular method according to one aspect of the present disclosure, however, is intended to include reference to a plurality of methods according to a plurality of aspects of the present disclosure.

The method according to one or more aspects of the present disclosure comprises the following steps:
(a) receiving, with a computing device, subject value data for the patient, the subject value data including and/or being indicative of (i) at least one troponin value, (ii) at least one demographic value, and (iii) at least one of a value for prior history of cardiac disease, a value of prior history of renal disease, an erythrocyte mean corpuscular hemoglobin value, and an electrolyte value;
(b) evaluating, with the computing device, the received subject value data of the patient based on a reference dataset indicative of reference subject values associated with one or more reference patients; and
(c) determining and/or assessing the risk of MACE based on the evaluation of the received subject value data of the patient of step (b).

Therein, the reference dataset is indicative of reference subject values including (i) at least one troponin value, (ii) at least one demographic value, and (iii) at least one of a value for prior history of cardiac disease, a value of prior history of renal disease, an erythrocyte mean corpuscular hemoglobin value, and an electrolyte value. In particular, the reference dataset may be indicative of reference subject values including (i) at least one troponin value, (ii) at least one demographic value, and (iii) a value for prior history of cardiac disease.

The subject value data may include subject values for the patient, which may be indicative of at least one troponin value, at least one demographic value, and at least one of the value for prior history of cardiac disease, the value of prior history of renal disease, the erythrocyte mean corpuscular hemoglobin value, and the electrolyte value.

A further aspect of the present disclosure relates to a computer-implemented method of determining and/or assessing a risk of MACE in a patient, for example a patient with prior history of cardiac disease and/or an indeterminate patient, the method comprising the steps of:
(a) receiving, with a computing device, subject value data for the patient, the subject value data including and/or being indicative of (i) at least one troponin value, (ii) at least one demographic value, and (iii) a value for prior history of cardiac disease;
(b) evaluating, with the computing device, the received subject value data of the patient based on a reference dataset indicative of reference subject values associated with one or more reference patients; and
(c) determining and/or assessing the risk of MACE based on the evaluation of the received subject value data of the patient of step (b).

Therein, the reference dataset is indicative of reference subject values including (i) at least one troponin value, (ii) at least one demographic value, and (iii) at least one of a value for prior history of cardiac disease, a value of prior history of renal disease, an erythrocyte mean corpuscular hemoglobin value, and an electrolyte value. In particular, the reference dataset may be indicative of reference subject values including (i) at least one troponin value, (ii) at least one demographic value, and (iii) a value for prior history of cardiac disease.

A further aspect of the present disclosure relates to a computer-implemented method of determining and/or assessing a risk of MACE in a patient, for example a patient with prior history of renal disease and/or an indeterminate patient, the method comprising the steps of:
(a) receiving, with a computing device, subject value data for the patient, the subject value data including and/or being indicative of (i) at least one troponin value, (ii) at least one demographic value, and (iii) a value for prior history of renal disease;
(b) evaluating, with the computing device, the received subject value data of the patient based on a reference dataset indicative of reference subject values associated with one or more reference patients; and
(c) determining and/or assessing the risk of MACE based on the evaluation of the received subject value data of the patient of step (b).

Therein, the reference dataset is indicative of reference subject values including (i) at least one troponin value, (ii) at least one demographic value, and (iii) at least one of a value for prior history of cardiac disease, a value of prior history of renal disease, an erythrocyte mean corpuscular hemoglobin value, and an electrolyte value. In particular, the reference dataset may be indicative of reference subject values including (i) at least one troponin value, (ii) at least one demographic value, and (iii) a value for prior history of cardiac and/or renal disease.

A further aspect of the present disclosure relates to a computer-implemented method of determining and/or assessing a risk of MACE in a patient, for example a patient with prior history of renal disease, a patient with prior history of cardiac disease and/or an indeterminate patient, the method comprising the steps of:
(a) receiving, with a computing device, subject value data for the patient, the subject value data including and/or being indicative of (i) at least one troponin value, (ii) at least one demographic value, and (iii) at least one of an erythrocyte mean corpuscular hemoglobin value and an electrolyte value;
(b) evaluating, with the computing device, the received subject value data of the patient based on a reference dataset indicative of reference subject values associated with one or more reference patients; and
(c) determining and/or assessing the risk of MACE based on the evaluation of the received subject value data of the patient of step (b).

Therein, the reference dataset is indicative of reference subject values including (i) at least one troponin value, (ii) at least one demographic value, and (iii) at least one of a value for prior history of cardiac disease, a value of prior history of renal disease, an erythrocyte mean corpuscular hemoglobin value, and an electrolyte value. In particular, the reference dataset may be indicative of reference subject values including (i) at least one troponin value, (ii) at least one demographic value, and (iii) a value for prior history of cardiac disease.

The inventors surprisingly found that taking at least one troponin value, at least one demographic value, and at least one of the value for prior history of cardiac disease, the value of prior history of renal disease, the erythrocyte mean corpuscular hemoglobin value, and the electrolyte value of the patient into consideration, the risk for MACE can be more accurately and reliably determined, for example when compared to determining the risk for MACE only based on the troponin value of the patient. In other words, the number of false positives and/or false negatives in determining the low risk for MACE and/or determining a high risk for MACE can be advantageously reduced.

It is noted that for reasons of simplicity, prognosing MACE in a patient, determining the risk of MACE in the patient, and/or determining a high risk for MACE may be used herein synonymously with determining whether the patient has MACE and/or with determining MACE in the patient.

Moreover, one or more of the at least one demographic value, the value for prior history of cardiac disease, the value of prior history of renal disease, the erythrocyte mean corpuscular hemoglobin value, and the electrolyte value of the patient may reflect or be indicative of a health state of the patient. Hence, by taking one or more of these values into consideration, the determination of MACE can be individualized to individual patients or patient sub-groups, which can further reduce the number of false positives and/or false negatives. Also, the time required for determining the risk of MACE can be significantly reduced. Accordingly, the present invention may significantly improve differentiation between patients having a high likelihood of experiencing a MACE event and those who are unlikely to experience a MACE event.

The present disclosure, therefore, can provide for an improved clinical decision support, for example allowing to efficiently, reliably and accurately risk stratify a patient as having a high or low risk for MACE. For instance, aspects of the present disclosure may facilitate earlier discharge of patients from hospitals for patient having a low risk of MACE, and earlier intervention for those patients who are more likely to experience a MACE event (and/or who are cardiac patients). In particular, unnecessary hospitalization of patients with non-critical disorders and anxiety among patients may be efficiently avoided or reduced. Accordingly, certain procedures of cardiac workflows and protocols can be avoided at hospitals, which can result in a better patient experience, improved utilization of healthcare resources and cost savings, all while maintaining a high safety profile.

Although not limited thereto, the present invention may be of particular advantage for determining and/or assessing the risk of MACE in patients, where the troponin level or value alone may not suffice to immediately and/or definitely rule in or rule out a patient as having a cardiac event, for example where the troponin level of the patient may fall into a so-called indeterminate range or zone of troponin values. Therein, the indeterminate range or zone of troponin values may refer to or denote a range of troponin values, based on which the patient cannot, or at least not with sufficient certainty or sufficiently high probability, be ruled in or out as being a cardiac patient or non-cardiac patient. As used herein, patients having troponin values in an indeterminate range of troponin values may also be referred to as an indeterminate patient or a patient belonging the group of indeterminate patients. Accordingly, one or more aspects of the present disclosure may, alternatively or additionally, relate to a method of determining a risk of MACE in an indeterminate patient. In particular, aspects of the present disclosure may allow to improve or maximize the risk stratification capabilities in the indeterminate group of patients, for example patients presenting with symptoms of ACS at a hospital or emergency department.

According to an embodiment, the method further comprises generating information indicative of the determined risk of MACE in the patient. Alternatively or additionally, the generated information may be indicative of the likelihood or probability for the patient having a health-relevant cardiac event and/or having MACE. In an example, the generated information may include an estimate of whether the patient suffers from or has a health relevant cardiac event and/or is at risk of MACE. Optionally, the computing device may determine or generate an output based on or including the generated information, which may for example be displayed at a user interface of the computing device.

Further, at least one troponin value of the patient and/or the one or more troponin values associated with one or more reference patients may be based on any type of one or more troponin tests performed on the patient and/or reference patient. For example, at least one troponin value of the patient and/or the one or more troponin values associated with one or more reference patients can relate to or be based on any one or more of a troponin I test (TnI), a high sensitivity troponin I test (hs-TnI), a troponin T test (TnT), and a high sensitivity troponin T test (hs-TnT).

As used herein, at least one troponin value of the patient and/or the one or more troponin values associated with one or more reference patients can relate to or be based on a measurement of troponin performed on the patient or reference patient, for example based on laboratory testing of sample material of the patient or reference patient. Accordingly, a troponin value can refer to a level or concentration of troponin at the time of assessment or testing. Alternatively or additionally, a troponin value can refer to a temporal change rate of troponin in the patient or reference patient and/or a change of troponin over time in the patient or reference patient. Therein the temporal change rate of troponin and/or the change of troponin over time may be based on a plurality of consecutively or sequentially determined levels or concentrations of troponin in the patient or reference patient.

In an embodiment, the method comprises receiving a plurality of consecutively or sequentially determined or measured troponin values for the patient, and computing a temporal change rate of troponin in the patient and/or determining a change of troponin in the patient over time based on the received plurality of troponin values. Alternatively or additionally, a plurality of consecutively or sequentially determined or measured troponin values associated with a reference patient may be used to compute a change of troponin in the reference patient over time.

In an exemplary embodiment, the method may comprise ruling in or ruling out the patient as at risk of MACE. Alternatively or additionally, the method may comprise ruling in or ruling out the patient as having a high risk of MACE. Therein, determining a risk of MACE in the patient may include determining a risk of MACE above or exceeding a risk threshold or cut-off value for the risk, e.g., an upper risk threshold or cut-off value. In other words, a patient may be ruled in as at risk for MACE based on determining a high risk or probability for MACE, for example a risk of MACE above about 60%, above about 70%, above about 80%, or above about 90%. Determining such risk for MACE may also be indicated when a patient has been diagnosed with an acute myocardial infarction or other health-relevant cardiac event, which the patient may potentially suffer from at the time of assessment of the risk of MACE.

Typically, patients who can be ruled in as cardiac patients or patients with high risk of MACE may have an elevated or abnormal troponin level or concentration, for example a troponin value greater than 50 ng/L, greater than 60 ng/L, greater than 70 ng/L or greater than 80 ng/L.

Alternatively or additionally, patients who can be ruled in as cardiac patients or patients with high risk of MACE may have an abnormal change or delta of troponin or troponin concentration over time, for example a change of troponin over time of greater than about 15 ng/L/hour, e.g. greater than 20 ng/L/hour, greater than 50 ng/L/hour, or greater than 100 ng/L/hour.

Alternatively or additionally, a patient may be ruled out because they are determined to have a low risk of MACE based on determining a risk of MACE is below a risk threshold or cut-off value for the risk, e.g., below a lower risk threshold or cut-off value. In other words, a patient may be ruled out as at low risk for MACE based on determining a low risk or probability for MACE, for example a risk of MACE below about 20%, 10%, 5%, 4%, 3%, 2%, or 1%.

Typically, patients who can be ruled out as cardiac patients and/or patients having a low risk of MACE may have a reduced or abnormal troponin level or concentration, for example a troponin value of less than 6 ng/L, less than 5 ng/L, less than 4 ng/L, less than 3 ng/L, or less than 2 ng/L.

Alternatively or additionally, patients who can be ruled out as cardiac patients and/or patients having a low risk of MACE may have a low change of troponin or troponin concentration over time, for example a change of troponin over time of less than about 1 ng/L/hour.

Further, the range or zone of indeterminate troponin values may range from a lower threshold (or rule-out cutoff) to an upper threshold (or rule-in cutoff) for the concentration of troponin. For example, the indeterminate range of troponin values may range from about 2 ng/L to about 80 ng/L.

Alternatively or additionally, the range or zone of indeterminate troponin values may range from a lower threshold (or rule-out cutoff) to an upper threshold (or rule-in cutoff) for the change of troponin over time, as described hereinabove. For example, the indeterminate range of troponin values may range from about 1 ng/L/hour to about 100 ng/L/hour, e.g. from about 1 ng/L/hour to about 15 ng/L/hour, as described hereinabove and hereinbelow.

The range of indeterminate troponin values or indeterminate patients may be exemplary illustrated as follows. A range of troponin values for a healthy patient population or group, i.e., non-cardiac patients, and a range of troponin values for cardiac patients may overlap in an indeterminate range of troponin values. Accordingly, it may be challenging to rule in or out a patient as cardiac patient, if the patient's troponin value is in the overlapping region. Thus, when only or primarily taking the troponin value into consideration, patients having troponin values falling in the indeterminate range may be assessed with an indeterminate risk of an emergent health-relevant cardiac event, which may not allow to accurately determine whether the corresponding patient is a cardiac patient or not. Risk of MACE is typically most difficult to determine within the described indeterminate population of indeterminate patients.

According to the method of one or more aspects of the present disclosure, the risk of MACE is determined based on or considering at least one troponin value, at least one demographic value, and at least one of the value for prior history of cardiac disease, the value of prior history of renal disease, the erythrocyte mean corpuscular hemoglobin value and the electrolyte value of the patient. Accordingly, the risk of MACE may be determined based on a plurality of subject value data or subject values of the patient. For example, compared to a determination of the risk of MACE based on the troponin value alone, the multi-parameter based determination of the risk for MACE, as described in the present disclosure, may allow for a more accurate, faster, more reliable and/or more individualized determination of the risk for MACE, in particular within indeterminate patients and/or patients having (current) troponin values falling in the indeterminate range of troponin values.

Examples of indeterminate patients or patients having troponin values in the indeterminate range or zone of troponin values, which may also be referred to as “grey area” for being ruled in or out as cardiac patients, can be patients with prior history of cardiac disease, for instance because these patients may have increased or abnormal troponin values, e.g., abnormal troponin concentrations and/or abnormal changes of troponin over time, caused by previously infarcted myocardial tissue. Other examples may include patients with prior history of renal disease, who typically show increased or abnormal troponin values, e.g. abnormal troponin concentrations and/or abnormal changes of troponin overtime, caused by kidney disease, kidney malfunction or kidney treatment. It is emphasized, however, that the present disclosure is not limited to indeterminate patients, nor limited to such as patients with prior history of cardiac and/or renal disease.

In the context of the present disclosure, at least one troponin value of the patient, which is received (in step (a) of the method) by the computing device and evaluated based on the reference dataset may refer to a current or recent troponin concentration in the patient, for example a troponin concentration determined in a recent troponin test performed on the patient. At least one troponin value of the patient may, for example, have been determined within a predetermined period of time prior to a time of assessment of the risk of MACE in the patient, such as within several hours, days, weeks or months prior to the time of assessment of the risk of MACE in the patient. Such current troponin value or concentration may be differentiated against or contrasted with a historic troponin value or concentration, which may refer to or denote a troponin value or concentration determined for the patient or the one or more reference patients in the past, for example prior to a predetermined time preceding the time of assessment for the risk of MACE in the patient. It is noted that the reference subject values, in particular the troponin values, associated with the one or more reference patients and indicated by the reference dataset can include one or both current and historic reference subject values, in particular troponin values.

Alternatively or additionally, a troponin value can refer to, include and/or be indicative of a temporal change rate of troponin (or troponin concentration) in the patient or reference patient and/or a change of troponin (or troponin concentration) over time in the patient or reference patient. Therein the temporal change rate of troponin and/or the change of troponin over time may be based on a plurality of consecutively or sequentially determined levels or concentrations of troponin in the patient or reference patient.

As used herein MACE may refer to a potential negative adverse outcome of a health-relevant cardiac event associated with or occurring in the patient. Examples of such health-relevant cardiac events can include one or more of acute coronary syndrome conditions or related negative health events such as, myocardial injury, cardiovascular death, ischemic cardiovascular events, heart failure, myocardial infarction, need for urgent revascularization, and/or stroke.

Further, as used herein, the reference patients, may refer to reference patients who had been previously diagnosed with a health relevant cardiac condition who are known to have experienced a negative health event (i.e. MACE) with the specified time period, such as patients who were diagnosed with one or more of the aforementioned health-relevant cardiac events in the past. For example, a corresponding patient or medical history of one or more reference patients may be reflected by or included in the reference dataset and/or the value for prior history of cardiac disease included reference subject values having experienced or similarly not experienced a MACE event.

As used herein, the value for prior history of cardiac disease may refer to or include an indicator specifying whether the patient was previously, e.g., prior to the determination of the risk of MACE in the patient, diagnosed with one or more e.g., health-relevant cardiac events. Accordingly, the value for prior history of cardiac disease of the patient, which may be received by the computing device (in step (a) of the method), may indicate whether a health-relevant cardiac event was previously determined in said patient. Alternatively or additionally, the value for prior history of cardiac disease included in the reference subject values indicated by the reference dataset may indicate whether a health-relevant cardiac event was previously determined in the corresponding reference patient.

For example, the value for prior history of cardiac disease of the patient and/or of the one or more reference patients may be a binary indicator with a first value indicating that the patient and/or the one or more reference patients were previously diagnosed with a health-relevant cardiac event, and a second value indicating that the patient and/or the one or more reference patients were not previously diagnosed with a health-relevant cardiac event. Example binary values may be “yes and no”, “0 and 1”, or any other binary indicator.

As used herein, the value for prior history of renal disease of the patient may refer to or include an indicator specifying whether the patient was previously, e.g., prior to the determination of the risk of MACE in the patient, diagnosed with one or more renal or kidney diseases. Optionally, the reference subject values may include a value for prior history of renal disease for one or more of the reference patients.

Alternatively or additionally, a current evidence of impaired renal function can be used. For example, a past clinical evidence of kidney disease and/or a current kidney malfunction may be utilized, e.g. using data obtained during the evaluation.

For example, the value for prior history of renal disease of the patient and/or of the one or more reference patients may be a binary indicator with a first value indicating that the patient and/or the one or more reference patients were previously diagnosed with a renal disease, and a second value indicating that the patient and/or the one or more reference patients were not previously diagnosed with a renal disease. Example binary values may be “yes and no”, “0 and 1”, or any other binary indicator.

Generally, the reference dataset may include, be representative of and/or indicative of medical records or health data associated with or related to one or more reference patients. In particular, the reference dataset may be indicative of at least the reference subject values, which may include (i) at least one troponin value, (ii) at least one demographic value, and (iii) the value for prior history of cardiac disease for the one or more reference patients, for example for each of the reference patients.

Optionally, the reference dataset may be indicative of one or more of a value for prior history of renal disease, an erythrocyte mean corpuscular hemoglobin value, and an electrolyte value, which may also be referred to herein as reference subject values.

Alternatively or additionally, the reference dataset may include or be indicative of one or more further reference subject values, further health data or further information, such as for example, data related to one or more of patient demographics, health insurance, admissions, encounters, diagnoses, therapies, surgeries, procedures, laboratory values, and laboratory test results.

In an exemplary implementation or embodiment, the reference dataset may be indicative of or include one or more predefined ranges of the demographic value and one or more thresholds for the at least one troponin value, wherein each predefined range of the demographic value may be associated with one of the one or more thresholds for the troponin value. Optionally, the step of evaluating the received subject value data of the patient based on the reference dataset may comprise determining, based on the demographic value of the patient, at least one of the predefined ranges of demographic values indicated by the reference dataset. Further, at least one threshold value associated with the determined range of demographic values in the reference dataset may be determined. Further, the method may comprise comparing the received troponin value of the patient to the determined at least one threshold value, which may for example include determining whether the troponin value of the patient reaches or exceeds an upper threshold for ruling in and/or a lower threshold for ruling out.

It should be noted that a threshold value for troponin may, in the context of the present disclosure, refer to, include and/or be indicative of a threshold for a troponin concentration and/or a threshold for a change in troponin values over time.

Optionally, the method and/or the step of determining the risk of MACE may comprise generating information indicative of a high risk, likelihood, or probability for MACE in response to or upon determining that the troponin value of the patient reaches or exceeds at least one threshold value. Alternatively, the method and/or the step of determining the risk of MACE may comprise generating information indicative of a low risk, likelihood, or probability for MACE in response to or upon determining that the troponin value of the patient falls below at least one threshold value.

Alternatively or additionally, the reference dataset may be indicative of or include one or more predefined ranges of the demographic value and one or more thresholds for at least one of an erythrocyte mean corpuscular hemoglobin value and an electrolyte value, wherein each predefined range of the demographic value may be associated with one of the one or more thresholds for at least one of the erythrocyte mean corpuscular hemoglobin value and the electrolyte value. Optionally, the subject value data of the patient may include at least one of an erythrocyte mean corpuscular hemoglobin value and an electrolyte value. Therein, the step of evaluating the received subject value data of the patient based on the reference dataset may comprise determining, based on the demographic value of the patient, at least one of the predefined ranges of demographic values indicated by the reference dataset. Further, at least one threshold value for at least one of the erythrocyte mean corpuscular hemoglobin value and the electrolyte value, which at least one threshold value may be associated with the determined range of demographic values in the reference dataset, may be determined. Further, the method may comprise comparing at least one of the erythrocyte mean corpuscular hemoglobin value and the electrolyte value of the patient to the determined at least one threshold value for at least one of the erythrocyte mean corpuscular hemoglobin value and the electrolyte value. This may include determining whether at least one of the erythrocyte mean corpuscular hemoglobin value and the electrolyte value of the patient reaches or exceeds the corresponding threshold value.

Optionally, the method and/or the step of determining the risk of MACE may comprise generating information indicative of a high risk, likelihood or probability for MACE in response to or upon determining that at least one of the erythrocyte mean corpuscular hemoglobin value and the electrolyte value of the patient reaches or exceeds the corresponding threshold value. Alternatively, the method and/or the step of determining the risk of MACE may comprise generating information indicative of a low risk, likelihood, or probability for MACE in response to or upon determining that at least one of the erythrocyte mean corpuscular hemoglobin value and the electrolyte value of the patient falls below the corresponding threshold value.

In an exemplary embodiment, one or more of the reference subject values may be determined by the computing based on one or more other reference subject values. In particular, the value for prior history of cardiac disease for one or more reference patients may be determined based on the at least one troponin value of the corresponding reference patient, as will also be discussed in more detail hereinbelow.

According to an embodiment, evaluating the received subject value data of the patient based on the reference dataset comprises analyzing the received subject value data using the reference dataset. For example, the received subject value data of the patient may be compared with the reference dataset.

The reference dataset may be stored on a data storage of the computing device and/or on at least one external data source, for example an external data source communicatively coupled with the computing device via a communication interface or circuitry of the computing device. Accordingly, the method may include, retrieving and/or accessing, with the computing device, the reference dataset on the data storage and/or on the external data source.

Alternatively or additionally, the reference dataset may be reflected by and/or implemented in a trained machine learning algorithm, such as for example a trained gradient boosting algorithm, or any other artificial intelligence-based (AI-based) algorithm.

Accordingly, the method according to one or more aspects of the present disclosure may be at least partly implemented as trained machine learning algorithm or any other artificial intelligence-based algorithm.

For example, the computing device may comprise a classifier or classifier circuitry, which may include a trained machine learning algorithm, a trained gradient boosting algorithm, or any other AI-based algorithm or circuitry. The classifier may be part of a control circuitry of the computing device or may be implemented as separate classifier circuitry in the computing device.

In an embodiment, the step of evaluating the received subject value data based on the reference dataset may be carried out by using a trained classifier, such as a trained machine learning algorithm, a trained gradient boosting algorithm, or other trained artificial intelligence-based algorithm. For instance, the received subject value data of the patient may be processed by means of the trained classifier of the computing device.

For example, the reference dataset may refer to or include data or parameters obtained during training of the classifier. Alternatively or additionally, the reference dataset may be used by the computing device during inference to evaluate and/or analyze the subject value data of the patient to determine the risk of MACE. Accordingly, the computing device may comprise a trained machine learning algorithm, a trained artificial intelligence-based algorithm, and/or a trained classifier for evaluating the received subject values of the patient based on the reference dataset.

Alternatively or additionally, one or more reference subject values associated with one or more reference patients may be used for training and/or used as training dataset of the machine learning algorithm, the artificial intelligence-based algorithm, and/or the classifier of the computing device. For instance, the trained machine learning algorithm, AI-based algorithm, and/or classifier may comprise a plurality of parameters, the value of said parameters being determined during training and comprised in the reference dataset. For instance, the computing device may be trained by using raw reference data associated with the one or more reference patients.

Accordingly, the evaluation of the received subject value data based on the reference dataset, which may for example include a comparison between the received subject value data of the patient with the reference dataset, may be carried out by processing the subject value data of the patient by means of the trained machine learning algorithm, AI-based algorithm and/or classifier by using the reference dataset.

According to an embodiment, the method may comprise determining and/or deriving at least a part of the reference dataset based on training a machine learning algorithm, an artificial intelligence-based algorithm, and/or a classifier of the computing device using raw reference data associated with one or more reference patients. The raw reference data may include one or more reference subject values, for example one or more of the reference subject values and/or one or more further reference subject values indicated by the reference dataset as described hereinabove and hereinbelow. For instance, the raw reference data may include one or more of at least one troponin value, at least one demographic value, a value for prior history of cardiac disease, a value for prior history of renal disease, an erythrocyte mean corpuscular hemoglobin value, and an electrolyte value of the one or more reference patients.

According to an embodiment, the step of determining the risk of MACE includes determining, computing and/or calculating a likelihood or probability that the patient will suffer from MACE within a predetermined period of time. Alternatively or additionally, the determined risk of MACE may be indicative of the likelihood or probability that the patient will suffer from MACE within a predetermined period of time.

According to an embodiment, the method further comprises generating information indicative of the determined risk of MACE in the patient. Alternatively or additionally, the generated information may be indicative of the likelihood or probability that the patient will suffer from MACE within a predetermined period of time. In an example, the generated information may include an estimate of whether the patient will suffer from MACE within a predetermined period of time.

According to an embodiment, the received subject value data of the patient include and/or are indicative of (i) at least one troponin value, (ii) at least one demographic value, and (iii) a value for prior history of cardiac disease of the patient.

According to a further embodiment, the received subject value data of the patient include and/or are indicative of (i) at least one troponin value, (ii) at least one demographic value, and (iii) a value for prior history of renal disease of the patient.

According to an embodiment, the received subject value data of the patient include and/or are indicative of (i) at least one troponin value, (ii) at least one demographic value, (iii) a value for prior history of cardiac disease of the patient, and (iv) a value for prior history of renal disease of the patient.

According to a further embodiment, the received subject value data of the patient include and/or are indicative of (i) at least one troponin value, (ii) at least one demographic value, and (iii) at least one of an erythrocyte mean corpuscular hemoglobin value and an electrolyte value of the patient.

According to a further embodiment, the received subject value data of the patient include and/or are indicative of (i) at least one troponin value, (ii) at least one demographic value, (iii) one or both of a value for prior history of cardiac disease and a value for prior history of renal disease, and (iv) one or both of an erythrocyte mean corpuscular hemoglobin value and an electrolyte value of the patient.

According to an embodiment, at least one or each of the subject value data and the reference subject values further comprise and/or are indicative of (iv) at least one of an erythrocyte mean corpuscular hemoglobin value and an electrolyte value. Alternatively or additionally, at least one or each of the subject value data and the reference subject values further comprise and/or are indicative of (v) a value for prior history of renal disease. Alternatively or additionally, at least one or each of the subject value data and the reference subject values further comprise and/or are indicative of (vi) a value for prior history of cardiac disease.

According to an embodiment, the reference dataset is indicative of and/or includes one or more reference subject values including one or more of a value of prior history of renal disease, an erythrocyte mean corpuscular hemoglobin value and an electrolyte value.

In an embodiment, the method further comprises determining one or more of the patient having a prior history of cardiac disease, and the patient having a prior history of renal disease. Alternatively or additionally, the method may comprise determining one or more of a value for prior history of cardiac disease and a value for prior history of renal disease of the patient. The patient having a prior history of cardiac disease, a prior history of renal disease, the value for the prior history of cardiac disease and/or the value for the prior history of renal disease can be determined by the computing device, for example, based on a user input indicative of the prior history of cardiac and/or renal disease. Alternatively or additionally, the computing device may be configured to process historic patient data indicative of a medical disease history of the patient. The historic patient data may be retrieved from a data storage of the computing device and/or from an external data source communicatively couplable to the computing device.

Generally, determining one or more of the patient having a prior history of cardiac disease, a prior history of renal disease, the value for the prior history of cardiac disease, and/or the value for the prior history of renal disease may allow to individually tailor the overall determination of the risk for MACE to the respective patient or patient group, for example by taking its medical disease history into account. Accordingly, the determination of the risk of MACE can be individualized and optimized to patients, which can further reduce the number of false positives and/or false negatives in the determination of the risk of MACE.

In an embodiment, determining one or more of the patient having a prior history of cardiac disease, a prior history of renal disease, the value for the prior history of cardiac disease, and/or the value for the prior history of renal disease includes receiving and/or processing historic patient data indicative of a medical disease history of the patient. The historic patient data may, for example, be stored on a data storage of the computing device and/or on one or more external data sources. Receiving the historic patient data may comprise retrieving and/or accessing the historic data stored at the data storage and/or the one or more external data sources.

Generally, the historic patient data may refer to or include one or more medical records or health data associated with and/or related to the patient. For example, the historic patient data may include data related to one or more of a current medication of the patient, a medication of the patient in the past, a clinical or medical treatment of the patient, a diagnostic procedure carried out on the patient, insurance, admissions, or encounters, a therapeutic procedure applied to the patient, a surgery carried out on the patient, laboratory test results or values, and the like.

In an embodiment, determining the patient having a prior history of renal disease and/or determining the value of prior history of renal disease includes determining, based on processing historic patient data indicative of a medical disease history of the patient, an estimated Glomerular Filtration Rate value, eGFR, and/or a creatinine value. Optionally, determining the determined eGFR and/or creatinine value may be compared to one or more predefined thresholds or threshold values for the eGFR and/or the creatinine value, which thresholds may optionally be indicated or defined by the reference dataset. For instance, it may be determined that the patient has a prior history of renal disease and/or a corresponding value of prior history of renal disease may be determined upon or based on determining that at least one eGFR and/or creatinine value reported for the patient or recorded in the historic patient data falls below the corresponding threshold for the eGFR and/or the creatinine value.

The eGFR value and/or the creatinine may, for example, be reported for the patient prior to the determination of the risk for MACE in said patient. Alternatively or additionally, the prior history of renal disease and/or the value for prior history of renal disease may be determined based on evaluating the historic patient data in terms of an eGFR value and/or a creatinine value, for example by determining an eGFR value and/or a creatinine value below the predetermined threshold value reported for the patient, for example reported prior to the determination of the risk for MACE in said patient. Generally, using one or more eGFR values and/or creatinine values recorded in the historic patient data may allow to reliably and quickly determine the prior history of renal disease, which can allow to further individualize and optimize the determination of the risk for MACE for the respective patient. It is noted, however, that one or more other indicators for renal disease may be used.

In an exemplary embodiment, the eGFR threshold value ranges from about 40 mL/min/1.73 m² to about 80 mL/min/1.73 m², for example from about 50 mL/min/1.73 m² to about 70 mL/min/1.73 m². In particular, the eGFR threshold value may be about 60 mL/min/1.73 m².

Alternatively or additionally, the creatinine threshold value may be about 1.3 mg/dL. These ranges or values for the eGFR and/or creatinine threshold described herein may allow for a reliable determination of a prior history of renal disease.

Other values or ranges of eGFR and/or creatinine, however, are also envisaged by the present disclosure.

Generally, normal levels of creatinine may be approximately 0.6 to 1.2 milligrams (mg) per deciliter (dL) in adult males and 0.5 to 1.1 milligrams per deciliter in adult females. Accordingly, creatinine values over about 1.3 mg/dL may be considered elevated, which could be related to e.g. kidney function or dehydration.

In an embodiment, determining the patient having a prior history of cardiac disease and/or determining the value for prior history of cardiac disease includes determining, based on processing historic patient data indicative of a medical disease history of the patient, a troponin value within a predetermined range and/or a troponin value above a predefined threshold. The troponin value may refer to a historic troponin value and may, for example, be reported for the patient prior to the determination of the risk for MACE in said patient.

Alternatively or additionally, the prior history of cardiac disease and/or the value for prior history of cardiac disease may be determined based on evaluating the historic patient data and determining one or more troponin values within the predetermined range and/or exceeding the predetermined threshold. Generally, using one or more troponin values recorded in the historic patient data and comparing these historic troponin values to the predetermined range and/or the threshold may allow to reliably and quickly determine the prior history of cardiac disease, which can allow to further individualize and optimize the determination of the risk for MACE for the respective patient. A troponin value falling within the predetermined range of values and/or exceeding the threshold may also be referred to as “abnormal” troponin value herein. Accordingly, the patient having a prior history of cardiac disease may be determined based on identifying one or more abnormal troponin values recorded for the patient, for example in the historic patient data. It is noted, however, that one or more other indicators for cardiac disease may be used including, for example, a previous diagnosis in the historic patient data.

In an exemplary embodiment, the predetermined range for the troponin value may be greater than 50 ng/L, greater than 60 ng/L, greater than 70 ng/L or greater than 80 ng/L. Such ranges or values for the troponin values reported for the patient may allow for a reliable determination of a prior history of cardiac disease in the patient. Other values or ranges of troponin, however, are also envisaged by the present disclosure.

Alternatively or additionally, the predetermined range for the troponin value may refer to an abnormal change of troponin or troponin concentration over time, for example a change of troponin over time greater than about 15 ng/L/hour, e.g. greater than 20 ng/L/hour, greater than 50 ng/L/hour, or greater than 100 ng/L/hour.

According to an embodiment, the method further comprises selecting and/or determining the reference dataset based on the determination of one or more of the patient having a prior history of cardiac disease, the value for prior history of cardiac disease, the patient having a prior history of renal disease, and the value for prior history of renal disease. Alternatively or additionally, the reference dataset may be selected and/or determined in response to, in accordance with and/or depending on the determination of one or more of the patient having a prior history of cardiac disease, the patient having a prior history of renal disease, the value for prior history of cardiac disease, and the value for prior history of renal disease. For instance, based on whether a prior history of cardiac or renal disease was determined for said patient, which may for example be indicated by the corresponding value for prior history of cardiac and/or renal disease, a corresponding reference dataset may be selected, invoked and/or loaded by the computing device. This can allow to further increase the accuracy in the determination of the risk for MACE and hence further reduce the number of false positives and/or false negatives.

By way of example, a first reference dataset may be selected, invoked and/or loaded upon determining that the patient has a prior history of cardiac disease and a second reference dataset may be selected, invoked and/or loaded by the computing device upon determining that the patient has a prior history of renal disease, wherein the first reference dataset may differ from the second reference dataset. For instance, the first and second reference datasets may differ in terms of one or more of the reference patients considered in the respective reference dataset, and/or one or more reference subject values indicated by the respective reference dataset.

In an example, each of the first and second reference dataset may be implemented as or included in a corresponding first or second machine learning algorithm, classifier and/or AI-based algorithm, and the corresponding algorithm or classifier may be selected, invoked, loaded and/or initiated by the computing device based on the determination of one or more of the patient having a prior history of cardiac disease, the value for prior history of cardiac disease, the patient having a prior history of renal disease, and the value for prior history of renal disease. For example, the first algorithm may be selected, invoked, initiated, and/or loaded upon determining that the patient has a prior history of cardiac disease and the second algorithm may be selected, invoked, initiated, and/or loaded by the computing device upon determining that the patient has a prior history of renal disease.

For example, one or more threshold values for one or more reference subject values may be indicated by the first reference dataset, which may differ at least partly from one or more threshold values for one or more reference subject values indicated by the second reference dataset. Alternatively or additionally, the first and second reference datasets may differ in terms of one or more further reference subject values indicated by the respective dataset, a sequence, in which the reference subject values and/or one or more further reference subject values are considered in the respective reference datasets, and one or more weighting factors applied to the reference subject values and/or one or more further reference subject values.

In an embodiment, the method further comprises deriving the reference dataset from raw reference data associated with one or more reference patients previously assessed for the risk for MACE. Analogue to the reference dataset, the raw reference data may include or be indicative of health data or information for the one or more reference patients, such as for example, data related to one or more of patient demographics, health insurance, admissions, encounters, diagnoses, therapies, surgeries, procedures, laboratory values, and laboratory test results. For example, the reference dataset may be derived from the raw reference data based on processing the raw reference data and/or based on using the raw reference data for training of a classifier of the computing device.

In an example, deriving the reference dataset may comprise one or more of combining data from a plurality of reference patients, filtering the raw reference data, for example for reference with patients with prior history of cardiac and/or renal disease, excluding one or more reference patients from the raw reference data, or otherwise processing the raw reference data to generate the reference dataset.

According to an embodiment, the reference dataset is indicative of and/or includes one or more threshold values for one or more of the reference subject values.

In an exemplary embodiment, selecting and/or determining the reference dataset includes selecting and/or determining one or more threshold values for one or more of the reference subject values based on the determination of one or more of the patient having a prior history of cardiac disease, the value for prior history of cardiac disease, the patient having a prior history of renal disease, and the value for prior history of renal disease. For example, the one or more threshold values for one or more of the reference subject values may be derived from the reference dataset or from raw reference data associated therewith. Alternatively or additionally, the one or more threshold values for one or more of the reference subject values may be selected and/or determined in response to, in accordance with and/or depending on the determination of one or more of the patient having a prior history of cardiac disease, the value for prior history of cardiac disease, the patient having a prior history of renal disease, and the value for prior history of renal disease. For instance, based on whether a prior history of cardiac or renal disease was determined for said patient and/or is indicated by the value for the prior history of renal and/or cardiac disease, one or more threshold values for one or more of the reference subject values may be selected, computed and/or calculated by the computing device.

Optionally, one or more threshold values for one or more reference subject values selected by the computing device upon determining prior history of cardiac disease may differ from one or more threshold values for one or more reference subject values selected by the computing device upon determining prior history of renal disease.

Further optionally, selecting and/or determining one or more threshold values for one or more of the reference subject values may include modifying, adjusting and/or altering one or more predefined threshold values for one or more of the reference subject values based on the determination of one or more of the patient having a prior history of cardiac disease, the value for prior history of cardiac disease, the patient having a prior history of renal disease, and the value for prior history of renal disease. For instance, one or more predefined threshold values may be stored at the computing device or retrieved from an external data source, and the computing device may adjust and/or modify the one or more predefined threshold values depending on whether the patient has a prior history of cardiac disease or a prior history of renal disease.

According to an embodiment, selecting and/or determining the reference dataset further comprises one or more of determining reference patients having a prior history of renal disease, and filtering the reference dataset and/or raw reference data for reference patients having a prior history of renal disease. For example, one or more reference patients having a prior renal disease may be identified and/or collected from the raw reference data to generate the reference dataset, for example based one or more values for prior history of cardiac and/or renal disease of one or more reference patients.

According to an embodiment, reference patients having a prior history of renal disease are determined based on determining an estimated Glomerular Filtration Rate value, eGFR, and/or a creatinine value, for example an eGFR value below a predefined eGFR threshold value and/or a creatinine value below a predefined creatinine threshold value reported for the corresponding reference patient. In an exemplary embodiment, the eGFR threshold value ranges from about 40 mL/min/1.73 m² to about 80 mL/min/1.73 m², for example from about 50 mL/min/1.73 m² to about 70 mL/min/1.73 m². In particular, the eGFR threshold value may be about 60 mL/min/1.73 m². Alternatively or additionally, the creatinine threshold value may be about 1.3 mg/dL. These ranges or values for the eGFR and/or creatinine threshold may allow for a reliable determination of a prior history of renal disease. Other values or ranges of eGFR and/or creatinine, however, are also envisaged by the present disclosure.

According to an embodiment, a threshold or threshold value indicative of renal disease is an eGFR threshold value, for example an eGFR from about 40 mL/min/1.73 m² to about 80 mL/min/1.73 m², for example from about 50 mL/min/1.73 m² to about 70 mL/min/1.73 m², in particular about 60 mL/min/1.73 m². Alternatively or additionally, the threshold or threshold value indicative of renal disease is creatinine threshold value, for example a creatinine value of about 1.3 mg/dL.

According to an embodiment, the reference dataset is indicative of reference subject values associated with reference patients who had been previously assessed for the risk for MACE and to whom a troponin value within a predetermined range and/or above a predetermined threshold, e.g. a predetermined range and/or threshold for the troponin concentration and/or a change of troponin over time, has been reported at the time of assessment. The time of assessment of the risk for MACE for one or more of the reference patients may be prior to the determination of the risk for MACE in the patient. In particular, the reference subject values may be associated with reference patients to whom an abnormal troponin value has been reported at the time of assessment.

According to an embodiment, the reference dataset is indicative of one or more threshold values for one or more of the reference subject values. In particular, the reference dataset may be indicative of one or more of a troponin threshold value (e.g., a threshold value for the troponin concentration and/or a threshold value for the change of troponin concentration over time), an erythrocyte mean corpuscular hemoglobin threshold value, and an electrolyte threshold value. Accordingly, the reference patients and/or the corresponding reference subject values may be reflected or included in the reference dataset by means of or based on the one or more threshold values. This may enable a fast, reliable, and accurate comparison of one or more of the received subject values of the patient with the one or more threshold values for the corresponding one or more reference subject values.

According to an embodiment, the method further comprises at least one of:
(a) evaluating at least one troponin value of the patient based on a troponin threshold value indicated by the reference dataset, and/or comparing at least one troponin value of the patient to a troponin threshold value indicated by the reference dataset;
(b) evaluating the at least one erythrocyte mean corpuscular hemoglobin value of the patient based on an erythrocyte mean corpuscular hemoglobin threshold value indicated by the reference dataset, and/or comparing at least one erythrocyte mean corpuscular hemoglobin value of the patient to an erythrocyte mean corpuscular hemoglobin threshold value indicated by the reference dataset; and
(c) evaluating the at least one electrolyte value of the patient based on an electrolyte threshold value indicated by the reference dataset, and/or comparing the at least one electrolyte value of the patient to an electrolyte threshold value indicated by the reference dataset.

For example, the received troponin value for the patient, which may refer to a current or recent troponin concentration determined or measured for the patient and/or a change of concentration of troponin determined or measured for the patient, may be intercompared with the corresponding troponin threshold value indicated by the reference dataset, and one or both the received at least one erythrocyte mean corpuscular hemoglobin value and the electrolyte value of the patient may be intercompared to a respective threshold value. This can allow to reliably rule in or out the patient as being a cardiac patient and/or having a higher or lower risk for MACE.

Optionally, the method and/or the step of determining the risk of MACE may comprise generating information indicative of a high risk, likelihood or probability for MACE in response to or upon determining that one or more of the erythrocyte mean corpuscular hemoglobin value, the electrolyte value, and at least one troponin value of the patient reaches or exceeds the corresponding threshold value. Alternatively, the method and/or the step of determining the risk of MACE may comprise generating information indicative of a low risk, likelihood, or probability for MACE in response to or upon determining that one or more of the erythrocyte mean corpuscular hemoglobin value, the electrolyte value, and at least one troponin value of the patient reaches or exceeds the corresponding threshold value falls below the corresponding threshold value.

According to an embodiment, the reference dataset is indicative of at least one troponin threshold value greater than 50 ng/L, greater than 60 ng/L, greater than 70 ng/L or greater than 80 ng/L. Alternatively or additionally, the reference dataset may be indicative of at least one troponin threshold value greater than about 15 ng/L/hour, e.g. greater than 20 ng/L/hour, greater than 50 ng/L/hour, or greater than 100 ng/L/hour. Such ranges or values for the troponin values reported for the one or more reference patients may allow for a reliable determination of a prior history of cardiac disease in the one or more reference patients. Other values or ranges of troponin, however, are also envisaged by the present disclosure.

According to an embodiment, the method further comprises determining a type of troponin test the received troponin value of the patient is based on, and determining the at least one troponin threshold value based on the determined type of troponin test. Accordingly, different troponin threshold values may be used depending on the type of troponin test performed on the patient to determine the troponin value received for the patient.

According to an embodiment, the method further comprises processing historic patient data indicative of a medical disease history of the patient, and determining the patient having a prior history of cardiac disease and/or determining the value for prior history of cardiac disease based on determining whether the patient was previously diagnosed with a cardiac disease.

According to an embodiment, the method further comprises processing historic patient data indicative of a medical disease history of the patient, and determining the patient having a prior history of renal disease and/or determining the value for prior history of renal disease based on determining an estimated Glomerular Filtration Rate value or creatinine value for the patient.

According to an embodiment, each of the subject value data and the reference subject values further comprise an electrolyte value, wherein the at least one electrolyte value of the subject value data includes at least one of a magnesium value and a potassium value.

According to an embodiment, the at least one electrolyte value of the subject value data includes at least one of a magnesium value and a potassium value. In other words, one or both of a magnesium value and a potassium value may be received for the patient. Such electrolyte values may have been determined for the patient by any appropriate clinical or medical test and provided to the computing device, for example via a user input, by storing the values on a data storage of the computing device and/or by storing the values on an external data source.

According to an embodiment, the reference dataset is indicative of at least one electrolyte threshold value, wherein the at least one electrolyte threshold value includes at least one of a magnesium threshold value and a potassium threshold value. In other words, one or both of a magnesium threshold value and a potassium threshold value may be indicated by or included in the reference dataset. For example, one or both of a magnesium value and a potassium value may be received for the patient and intercompared with one or both of the magnesium threshold value and the potassium threshold value indicated by the reference dataset.

According to an embodiment, the magnesium threshold value is about 1.6 mg/dL to about 2.0 mg/dL, for example about 1.8 mg/dL. Alternatively or additionally, the potassium threshold value is about 2.3 mg/dL to about 2.7 mg/dL, for example about 2.5 mg/dL. Such threshold values may allow to reliably determine the risk for MACE in the patient, in particular when considered in addition to the troponin value of the patient.

According to an embodiment, the at least one demographic value includes at least one of a gender value, a racial value, and an age value. The gender value may be indicative of the gender of the patient or reference patient, the racial value may be indicative of a race of the patient or reference patient, and the age value may be indicative of the patient's or reference patient's age. By taking one or more of the aforementioned demographic values into consideration, gender-related, race-related and/or age-related influences on the determination of the risk for MACE can be accounted for, which can allow to further individualize or optimize the risk determination.

For instance, based on the demographic value, gender-matched, age-matched, and/or race-matched troponin threshold values may be determined by the computing device and/or a corresponding reference dataset indicative of the one or more gender-matched, age-matched, and/or race-matched troponin threshold values may be determined by the computing device to evaluate the received subject value data of the patient and/or to determine the risk of MACE.

According to an embodiment, the method further comprises determining a gender of the patient based on the demographic value, e.g., the gender value comprised by the demographic value. Alternatively or additionally, the method may comprise classifying the patient into an age group of a plurality of predefined age groups based on the demographic value, e.g. the age value comprised by the demographic value. Alternatively or additionally, the method may comprise classifying the patient into a race group of a plurality of predefined race groups based on the demographic value, e.g., the race value comprised by the demographic value. Alternatively or additionally, the method may comprise selecting and/or determining the reference dataset based on the at least one demographic value received for the patient.

According to an embodiment, the method further comprises receiving further subject value data for the patient, the further subject value data including one or more of a ratio of oxyhemoglobin to hemoglobin, a ratio of neutrophils per leukocytes, a ratio of nucleated erythrocytes to leukocyte, and a ratio of eosinophils to leukocytes. Further, the method comprises evaluating the further subject value data based on one or more further reference subject values indicated by the reference dataset, for example by comparing the further subject value data to one or more further reference subject values indicated by the reference dataset. Therein, the one or more reference subject values may be indicative of and/or include one or more of a ratio of oxyhemoglobin to hemoglobin, a ratio of neutrophils per leukocytes, a ratio of nucleated erythrocytes to leukocyte, and a ratio of eosinophils to leukocytes.

Optionally, the method may further comprise generating information and/or a score indicative of the determined risk of MACE in the patient based on the evaluation of the further subject value data or the comparison thereof with the reference dataset. Alternatively or additionally, generating information indicative of the determined risk of MACE in the patient may comprise computing and/or calculating the score.

The one or more further reference subject values may be stored on a data storage of the computing device and/or at one or more external data sources. It is noted that any one or more of the aforementioned further subject values or corresponding further reference subject values may be taken into consideration for the determination of the risk for MACE, optionally in a predefined sequence or order.

Optionally, a result of the comparison of one or more of the of the aforementioned further subject values with corresponding further reference subject values may be weighted, for example to account for different levels of importance or relevance of the further subject values may have on the determination of the risk for MACE in the patient.

According to an embodiment, the method further comprises computing and/or calculating a score indicative of a likelihood for the patient having MACE, e.g., within a predetermined period of time, based on the evaluation of the received subject value data of the patient using the reference dataset, for example based on the comparison between the received subject value data of the patient and the reference dataset.

According to an embodiment, the method further comprises computing and/or calculating a score indicative of a likelihood or probability for the patient having MACE, for example by evaluating at least one troponin value of the patient based on at least one troponin threshold value indicated by the reference dataset, and based on evaluating at least one of the erythrocyte mean corpuscular hemoglobin value and the electrolyte value of the patient based on at least one of an erythrocyte mean corpuscular hemoglobin threshold value and an electrolyte threshold value indicated by the reference dataset.

For example, at least one troponin value of the patient may be compared with at least one troponin threshold value indicated by the reference dataset, and at least one of the erythrocyte mean corpuscular hemoglobin value and the electrolyte value of the patient may be compared with at least one of an erythrocyte mean corpuscular hemoglobin threshold value and an electrolyte threshold value indicated by the reference dataset. Accordingly, the score indicative of the likelihood for MACE in the patient may be computed based on a result of the comparison of at least one troponin value of the patient to at least one troponin threshold value, and based on the result of the comparison of one or both of the erythrocyte mean corpuscular hemoglobin value and the electrolyte value of the patient with one or both of the erythrocyte mean corpuscular hemoglobin threshold value and the electrolyte threshold value.

Generally, the score may refer to an indicator reflecting an estimate that the patient has MACE. The score can be a numerical score on an arbitrary scale, such a scale between zero and one or 0% and 100%. Alternatively or additionally, the score may be a graphical indicator or any other appropriate indicator. Optionally, the score may be displayed, for example at a user interface of the computing device or any other device.

According to an embodiment, computing the score includes:
(a) determining a first partial score based on the comparison of the at least one troponin value of the patient to the at least one troponin threshold value;
(b) determining at least one second partial score based on the comparison of the at least one of the erythrocyte mean corpuscular hemoglobin value and the electrolyte value of the patient with the at least one of the erythrocyte mean corpuscular hemoglobin threshold value and the electrolyte threshold value; and
(c) computing the score indicative of the likelihood for MACE based on the determined first partial score and the determined at least one second partial score.

Therein, the first partial score may be indicative of a first partial likelihood for the patient suffering from MACE and the second partial score may be indicative of a second partial likelihood for the patient suffering from MACE. The first and second partial likelihoods may be computed by the computing device or may be predefined and, for example, selected by the computing device based on the comparison.

Optionally, one or more mathematical operations may be applied to the first and second partial scores to generate the score. In a non-limiting example, the partial scores may be added to calculate the score. Alternatively, the partial scores may be intercompared and the higher or lower partial score may be selected as a revised score. Optionally, different weighting factors may be applied to the first and second partial scores to compute the score. Also, an average of the partial scores may be computed.

In an example, the method may comprise determining, for each received subject value and/or further subject value, a partial score based on comparing each received subject value and/or further subject value with a corresponding threshold value indicated by the reference dataset, and computing the score based on the partial scores determined for each received subject value and/or further subject value. For example, the partial scores may be added to compute the score or any other mathematical operation may be applied to the partial scores, as described above.

According to a further aspect of the present disclosure, there is provided a computer program, which, when executed by one or more processors of a computing device, instructs the computing device to perform steps of one or more methods according to one or more aspects of the present disclosure, as described hereinabove and hereinbelow.

According to a further aspect of the present disclosure, there is provided a computer-readable medium, e.g., a non-transitory computer-readable medium, storing a computer program, which, when executed by one or more processors of a computing device, instructs the computing device to perform steps of one or more methods according to one or more aspects of the present disclosure, as described hereinabove and hereinbelow.

A further aspect of the present disclosure relates to a computing device configured to perform steps of one or more methods according to one or more aspects of the present disclosure, as described hereinabove and hereinbelow. Generally, the computing device may refer to a clinical decision support system or device for determining the likelihood of MACE in a patient, for example a patient with prior history of cardiac and/or renal disease.

A further aspect of the present disclosure relates to use of such computing device for determining the likelihood of MACE in a patient.

The computing device may be embodied as any type of data processing device, such as a smartphone, a desktop computer, a server, a server network, a cloud computing network, or the like.

The computing device may include one or more processors for data processing and at least one data storage for storing data, such as the reference dataset, the received subject values, raw reference data, historic patient data, one or more threshold values for one or more subject values, or any other data.

Alternatively or additionally, a computer program or software instructions may be stored on the data storage, which, when executed by one or more processors of the computing device, instructs the computing device to perform steps of one or more methods according to one or more aspects of the present disclosure, as described hereinabove and hereinbelow

Optionally, the computing device may comprise at least one communication circuitry or interface for communicatively coupling the computing device to one or more external data sources that may optionally store data, such as the reference dataset, the subject values, raw reference data, historic patient data, one or more threshold values for one or more subject values, or any other data.

These and other aspects of the disclosure will be apparent from and elucidated with reference to the appended figures, which may represent exemplary embodiments.

### BRIEF DESCRIPTION OF THE DRAWINGS

The subject-matter of the present disclosure will be explained in more detail in the following with reference to exemplary embodiments which are illustrated in the attached drawings, wherein:

FIG. 1 shows a computing device for determining a risk for MACE in a patient according to an exemplary embodiment; and

FIG. 2 shows a flow chart illustrating a method of determining a risk for MACE in a patient according to an exemplary embodiment; and

FIG. 3 shows exemplary histograms indicative of two populations of subjects (a “reference population” and a “disease population”) to illustrate steps of a method of determining a risk of MACE.

The figures are schematic only and not true to scale. In principle, identical or like parts are provided with identical or like reference symbols in the figures.

### DETAILED DESCRIPTION OF EXEMPLARY EMBODIMENTS

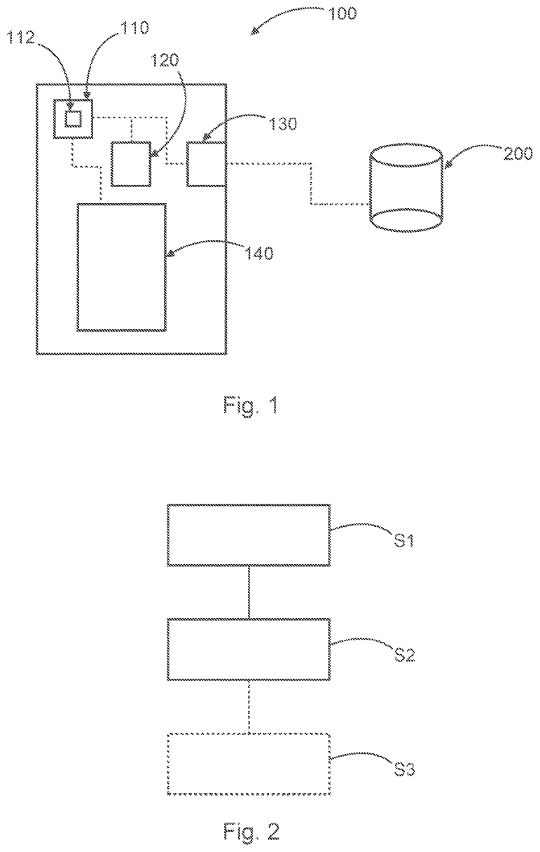

FIG. 1 shows a computing device 100 or clinical decision support system 100 for determining the risk for MACE in a patient according to an exemplary embodiment.

The computing device 100 comprises a processing circuitry 110 or control circuitry 110 with one or more processors 112 for data processing. Optionally, the processing circuitry 110 or control circuitry 110 may include a classifier or a classifier circuitry. The computing device 100 further comprises at least one data storage 120 for storing data.

The exemplary computing device 100 of FIG. 1 further comprises at least one communication circuitry or interface 130 for communicatively coupling the computing device 100 to one or more external data sources 200 that may optionally store data and/or provide data to the computing device 100. The communication circuitry 130 may be configured for wired or wireless communication with the at least one external data source 200. It should be noted that the computing device 100 may comprise a plurality of communication circuits 130 or interfaces 130 for communicatively coupling the computing device 100 to a plurality of different external data sources 200.

The one or more external data sources 200 may for example be associated with one or more external servers communicatively coupled to the computing device 100, for example via the Internet, a LAN connection, a wireless connection or a wired connection. For example, the computing device 100 may be communicatively couplable to a hospital information system, a laboratory information system, a server of a health care provider, or any other server.

As discussed in detail hereinabove and hereinbelow, the computing device 100 may be configured to determine the risk of MACE in a patient. In particular, the computing device 100 is configured to receive subject value data for the patient, the subject value data including and/or being indicative of subject values, which may include (i) at least one troponin value, (ii) at least one demographic value, and (iii) at least one of a value for prior history of cardiac disease, a value of prior history of renal disease, an erythrocyte mean corpuscular hemoglobin value, and an electrolyte value for the patient. One or more of these subject values may be received by the computing device 100 based on retrieving the one or more subject values from the data storage 120 and/or from one or more external data sources 200.

Further, the computing device 100 is configured to evaluate the received subject value data or corresponding subject values included in or indicated by the subject value data of the patient based on a reference dataset indicative of reference subject values associated with one or more reference patients. For example, the computing device 100 may be configured to compare the received subject value data or corresponding subject values included in the subject value data of the patient with a reference dataset indicative of reference subject values associated with one or more reference patients, for example reference patients who had been previously assessed for a risk of MACE. Therein, the reference dataset may be indicative of reference subject values including (i) at least one troponin value, (ii) at least one demographic value, and (iii) a value for prior history of cardiac disease. Optionally, the reference data set may be indicative of at least one of an erythrocyte mean corpuscular hemoglobin value and an electrolyte value. The reference dataset may be retrieved by the computing device 100 from the data storage 120 and/or from one or more external data sources.

Optionally, the computing device 100 may be configured to determine the reference dataset, for example based on processing raw reference data associated with one or more reference patients previously determined to have a high risk of MACE. Alternatively or additionally, the raw reference data may be used for training a classifier, machine learning algorithm and/or AI-based algorithm of the computing device 100 to determine the reference dataset, as described hereinabove.

The raw reference data and/or the reference dataset may include or be indicative of health data or information for the one or more reference patients, such as for example, data related to one or more of patient demographics, health insurance, admissions, encounters, diagnoses, therapies, surgeries, procedures, laboratory values, and laboratory test results.

The computing device 100 may for example be configured to derive the reference dataset from the raw reference data. For instance, the computing device may be configured to combine or merge data from a plurality of reference patients, to filter the raw reference data, for example for reference with patients with prior history of cardiac and/or renal disease, to exclude one or more reference patients from the raw reference data, or otherwise process the raw reference data to generate the reference dataset. Optionally, raw reference data from different data sources may be combined or used by the computing device 100 to generate the reference dataset.

For example, the computing device 100 may be configured to determine one or more threshold values for one or more reference subject values, which may be used for comparison with one or more subject values received for the patient. Accordingly, the reference dataset may be indicative of one or more threshold values for one or more reference subject values. For instance, the reference dataset may be indicative of one or more of a troponin threshold value, an erythrocyte mean corpuscular hemoglobin threshold value, an electrolyte threshold value, a magnesium threshold value, a potassium threshold value, and at least one further threshold value for at least one further subject value.

The computing device 100 may further be configured to compare the at least one troponin value of the patient to a troponin threshold value indicated by the reference dataset, to compare the at least one erythrocyte mean corpuscular hemoglobin value of the patient to an erythrocyte mean corpuscular hemoglobin threshold value indicated by the reference dataset, and/or to compare the at least one electrolyte value of the patient to an electrolyte threshold value indicated by the reference dataset.

Further, the computing device 100 may be configured to receive further subject value data for the patient, the further subject value data including one or more further subject values for the patient, such as for example a ratio of oxyhemoglobin to hemoglobin, a ratio of neutrophils per leukocytes, a ratio of nucleated erythrocytes to leukocyte, and a ratio of eosinophils to leukocytes. The computing device 100 may further be configured to compare one or more further subject values to one or more further reference subject values of the reference dataset, the one or more reference subject values being indicative of one or more of a ratio of oxyhemoglobin to hemoglobin, a ratio of neutrophils per leukocytes, a ratio of nucleated erythrocytes to leukocyte, and a ratio of eosinophils to leukocytes.

Generally, the computing device 100 may be configured to determine the risk of MACE in the patient based on evaluating one or more subject values and/or further subject values for the patient, for example based on comparing the (further) subject values to one or more (further) reference subject values indicated by the reference dataset.

Optionally, other or additional variables and/or indicators may be used by the computing device 100 to determine the risk of MACE in the patient. For instance, one or more demographic values or variables, such as age, gender, and race may be used by the computing device 100 for determining the risk of MACE. Alternatively or additionally, clinical variables, such as a type of a disorder or comorbidity, a finding, a symptom, a procedure performed on the patient, a laboratory finding, a medication, and a disease indicator, such as a diabetes indicator, a hypertension indicator or an indicator for abnormal diastolic, may be used by the computing device 100 for determining the risk of MACE. Alternative or additional variables may indicate abnormal lipids, abnormal cholesterol (LDL or HDL cholesterol), a catheterization of the patient or any other procedure or treatment performed on the patient.

In the following, non-limiting examples of subject values, further subject values, indicators, and/or variables that may be taken into consideration by the computing device 100 for determining the risk of MACE are summarized: Gender, blood pressure, age, age group, erythrocyte mean corpuscular hemoglobin, atrial fibrillation, pH value, glomerular filtration rate, Oxyhemoglobin per Hemoglobin, neutrophils per 100 leukocytes, electrolyte value, magnesium value, potassium value, erythrocytes nucleated per 100 leukocytes, abnormal systolic, natriuretic peptide, urea nitrogen, electrocardiogram, anion gap, eosinophils per 100 leukocytes, chronic obstructive lung disease, chemical metabolic function tests, and partial thromboplastin time. Although not limited thereto, one or more of the aforementioned indicators may be particularly useful for determining the risk of MACE in a patient with prior history of cardiac disease.

Other non-limiting examples of subject values, further subject values, indicators, and/or variables that may be taken into consideration by the computing device 100 for determining the risk of MACE are summarized in the following: Congestive heart failure, abnormal electrolyte, abnormal magnesium, abnormal potassium, electrocardiogram, race, atrial fibrillation, gender, urinary tract infectious disease, age, age group, erythrocyte mean corpuscular hemoglobin, glomerular filtration rate, anemia, end stage renal disease, acute renal failure syndrome, abnormal phosphate, hypertension, pH value, abnormal systolic, pneumonia, hypothyroidism, hypoglycemic events, chemical metabolic function tests, coronary artery bypass grafting, and neutrophils per 100 leukocytes. Although not limited thereto, one or more of the aforementioned indicators may be particularly useful for determining the risk of MACE in a patient with prior history of renal disease.

Optionally, the computing device 100 may be configured to determine one or more of the patient having a prior history of cardiac disease, the patient having a prior history of renal disease, the value for prior history of cardiac disease, and the value for prior history of renal disease, for example based on processing historic patient data indicative of a medical disease history of the patient.

For instance, the computing device 100 may be configured to determine that the patient has a prior history of renal disease based on determining an estimated Glomerular Filtration Rate value, eGFR, and/or a creatinine value, e.g., an eGFR value below a predefined eGFR threshold value and/or a creatinine value below a predefined creatinine threshold value, recorded for the patient in the historic patient data.

Alternatively or additionally, the computing device 100 may be configured to determine that patient has a prior history of cardiac disease based on processing historic patient data indicative of a medical disease history of the patient and determining a troponin value within a predetermined range and/or above a predetermined threshold value for troponin reported for the patient.

In an example implementation, the computing device 100 may be configured to select and/or determine the reference dataset based on the determination of one or more of the patient having a prior history of cardiac disease, the patient having a prior history of renal disease, the value for prior history of cardiac disease, and the value for prior history of renal disease. Selecting the reference dataset may, for example, include selecting one or more threshold values for one or more of the reference subject values based on the determination of one or more of the patient having a prior history of cardiac disease, the patient having a prior history of renal disease, the value for prior history of cardiac disease, and the value for prior history of renal disease. Alternatively or additionally, one or more further reference subject values may be selected by the computing device 100 and/or a sequence, in which these are considered, may be determined by the computing device 100 based on the determination of one or more of the patient having a prior history of cardiac disease, the patient having a prior history of renal disease, the value for prior history of cardiac disease, and the value for prior history of renal disease. Alternatively or additionally, selecting the reference dataset may comprise determining reference patients having a prior history of renal disease and/or filtering the reference dataset or raw reference data for reference patients having a prior history of renal disease.

In an example implementation, one or more of the aforementioned subject values subject value data, further subject values, further subject value data, indicators, and/or variables may be received for the patient and evaluated based on or against the reference dataset. The reference dataset may include one or more threshold values, against which one or more of the aforementioned subject values, further subject values, indicators, and/or variables may be compared to determine the risk of MACE.

Based on one or more of such comparisons, a score and/or information indicative of a likelihood for or risk of the patient suffering from MACE may be determined by the computing device 100. For instance, for each subject value or variable received for the patient and evaluated based on the reference dataset, a definable or predetermined partial score indicative of a partial likelihood for or risk of MACE may be computed, and the score determined by the computing device may be determined based on the partial scores. For example, partial scores can be added, averaged, weighted, or otherwise merged to generate the score.

The computing device 100 further includes a user interface 140 for receiving one or more user inputs. For instance, one or more subject values or other data may be provided to the computing device 100 via the user interface 140.

The user interface 140 may be configured to provide or output information to a user. For example, the computing device 100 may be configured to display the score and/or generated information indicative of the likelihood or risk of MACE and/or one or more partial scores at the user interface 140.

FIG. 2 shows a flow chart illustrating a method of determining a risk of a MACE in a patient according to an exemplary embodiment, for example using a computing device 100 as described with reference to FIG. 1. The method illustrated in FIG. 2 may, for example, relate to a method of determining a risk of a MACE in a patient with prior history of renal disease, prior history of cardiac disease, and/or in an indeterminate patient.

Step S1 comprises receiving, with the computing device 100, subject value data for the patient. The subject value data include and/or are indicative of subject values, which may include (i) at least one troponin value, (ii) at least one demographic value, and (iii) at least one of a value for prior history of cardiac disease, a value of prior history of renal disease, an erythrocyte mean corpuscular hemoglobin value and an electrolyte value for the patient. One or more of these subject values may be received by the computing device 100 based on retrieving the one or more subject values from the data storage 120 and/or from one or more external data sources 200.

Optionally, step S1 may comprise determining one or more of the patient having a prior history of cardiac disease, the value for prior history of cardiac disease, the patient having a prior history of renal disease, and the value of prior history of renal disease, as described hereinabove.

Step S2 comprises evaluating the received subject value data or corresponding subject values included in or indicated by the subject value data of the patient based on a reference dataset indicative of reference subject values associated with one or more reference patients. For example, the computing device 100 may be configured to compare the received subject value data or corresponding subject values included in or indicated by the subject value data of the patient with a reference dataset indicative of reference subject values associated with one or more reference patients, for example reference patients who had been previously assessed for MACE. Therein, the reference dataset may be indicative of reference subject values including (i) at least one troponin value, (ii) at least one demographic value, and (iii) a value for prior history of cardiac disease. Optionally, the reference data set may be indicative of at least one of an erythrocyte mean corpuscular hemoglobin value and an electrolyte value.

The reference dataset may be retrieved by the computing device 100 from the data storage 120 and/or from one or more external data sources. Alternatively or additionally, the reference dataset may be determined, selected, loaded and/or invoked by the computing device 100, e.g. based on the received subject value data of the patient.

It is noted that one or more further optional steps, as described hereinabove in the summary part and with reference to FIG. 1, may be performed for determining the risk of MACE in the patient. For example, in an optional step S3 a score and/or information indicative of a likelihood for the patient having or suffering from MACE may be computed, which can optionally be displayed at a user interface 140 of the computing device.

In an illustrative example, patient A and patient B may be assessed for the risk of MACE in accordance with the present disclosure. Patient A may be a 50-year-old male and patient B may be an 80-year-old female. The computing device 100 may receive a demographic value for each of patient A and B, which can include a corresponding age value and gender value, based on which the computing device 100 can determine the age and gender of patients A and B. The computing device 100 may further determine a troponin threshold value for each of patients A and B, based on the respective demographic value of patient A and B, for example based on the age value and/or the gender value.

Optionally, the computing device 100 may further determine a reference dataset for each of patients A and B, each reference dataset being indicative of the respective troponin threshold for patient A or B. For instance, the reference dataset and/or troponin threshold value determined for patient A may be lower (or greater) than the troponin threshold value determined for patient B. Further, the computing device 100 may receive a current or recent troponin value for each of patients A and B and compare the troponin values with the respective troponin threshold values for patient A and B. If the received troponin value exceeds or reaches the troponin threshold value for patient A or B, the computing device 100 may generate information and/or a score indicating a high risk of MACE for the respective patient A or B and/or indicating patient A or B as cardiac patient. Alternatively, if the received troponin value is below the troponin threshold value for patient A or B, the computing device 100 may generate information and/or a score indicating a low risk of MACE for the respective patient A or B and/or indicating patient A or B as non-cardiac patient.

Similarly, one or more further subject values of the patient, such as an electrolyte value and/or an erythrocyte mean corpuscular hemoglobin value, a race value, or other subject values, as described herein, may be used by the computing device to determine a corresponding reference dataset for evaluating the received subject value data.

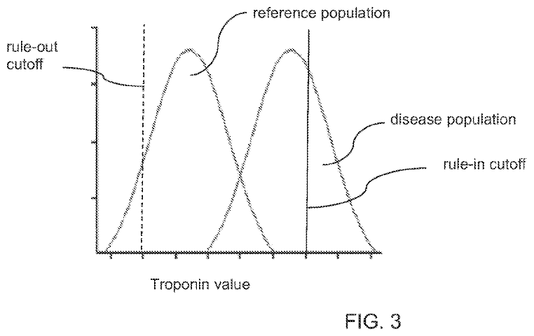

FIG. 3 illustrates two exemplary histograms indicative of two populations of patients to illustrate steps or aspects of a method of determining the risk of MACE. The first population (indicated by the right-most histogram) includes or is indicative of a “disease population” or “disease patients”, which were identified because they were determined to suffer from a health-relevant cardiac event. The second population (indicated by the leftmost histogram) includes or is indicative of a “reference population” or “reference patients”. The population of reference patients may be selected according to any criteria determined herein (e.g., age-matched, demographic-matched, geographically matched, weight-matched, height-matched, gender-matched, etc.). The histograms in FIG. 3 are shown as the number of patients or subjects (y-axis) versus troponin value or concentration (x-axis) in arbitrary units.

In addition, the two histograms could be used to calculate a risk of MACE for a patient in the left most histogram representing those which did not experience a (negative) health-relevant cardiac event or MACE, with the right most histogram indicative of those patients which did experience a (negative) health-relevant cardiac event, outcome or MACE.

As used herein, the term “presenting” or “presentation” generally refers to an assessment of a subject or patient, for example, when the subject or patient first arrives at the emergency department and is evaluated by a clinician, such as an emergency department physician or nurse. “Presenting” or “presentation” may also include subsequent assessments of the subject or patient, for example, when the concentration of troponin (or troponin value) in a sample taken from the subject or patient does not clearly fall above the rule-in cutoff or below the rule-out cutoff (also referred to as troponin thresholds or troponin threshold values herein), as discussed herein. In such situations, the subject or patient may require one or more additional assessments. Such additional assessments may be taken at time intervals determined to provide the most relevant clinical information. In one example, one, two, three, and/or more additional assessments may be following initial presentation, where one, two, three, and/or more additional samples may be taken from the subject to assess whether the subject or patient is exhibiting troponin concentrations or values that are characteristic of a disease population of reference patients (also referred to as disease reference patients) or if the patient is exhibiting troponin concentrations or values that are characteristic of a healthy population of reference patients (also referred to as healthy reference patients). In other words, additional samples may be taken from the patient to assess whether changes in troponin concentration over time indicate a progression of the patient toward one cutoff or the other.

Thus, for example, when a patient is presenting with symptoms suggestive of ACS arrives at an emergency department, a clinician may take a sample from the patient and compare the concentration of troponin in the sample with a reference troponin value The troponin concentration or value in the sample, relative to the reference troponin value (for example, the rule-in cutoff or threshold and/or the rule-out cutoff or threshold concentrations), may be used to determine whether the patient is more likely to be suffering from a health-relevant cardiac event or not; that is, whether the patient has a troponin value or concentration above the rule-in cutoff or threshold, indicating that the patient is more likely to be suffering from a health-relevant cardiac event or whether the patient has a troponin concentration or value below the rule-out cutoff or threshold, indicating that the patient is likely not suffering from a health-relevant cardiac event. Exemplary rule-in and rule-out cutoffs (or troponin threshold values) are shown in FIG. 3 by vertical lines.

Those of skill in the art will recognize that the reference patients or reference population may be matched for as many characteristics of the disease reference patients or disease reference population as possible or desired, save for the disease itself. Thus, for example, the reference patients or population, represented in one exemplary aspect in FIG. 3, may be age-matched, demographic-matched, geographically matched, weight-matched, height-matched, and/or gender-matched to the expected disease reference patients or population but not matched for the disease in question. Thus, for example, the healthy reference patients or population may include patients that are sex-matched and age-matched for the patient, for whom the risk of MACE is determined.

FIG. 3 further shows an exemplary rule-in cutoff or threshold; patients having a troponin value greater than the value of the rule-in cutoff indicates they are likely to be suffering from a health-relevant cardiac event. FIG. 3 further shows an exemplary rule-out cutoff or threshold; patients having a troponin value lower than the rule-out cutoff indicates they are likely not to be suffering from a health-relevant cardiac event. When a patient presenting symptoms of acute coronary syndrome arrives at an emergency department, a clinician may take a sample from the patient and compare the concentration or value of troponin in the sample with the rule-out cutoff troponin value or concentration and/or the rule-in cutoff troponin value concentration to determine the risk of MACE in the patient. Examples of rule-out cutoff troponin values or concentrations and rule-in cutoff troponin values or concentrations at presentation are shown in Table 1.

TABLE 1

| | Troponin value at presentation |
|---|---|
| Rule-out cutoff | <4 ng/L |
| Indeterminate | (Neither ruled-in nor ruled-out) |
| Rule-in cutoff | >50 ng/L |

In the example set forth in Table 1, the rule-out cutoff troponin value or concentration at presentation is less than 4 ng/L, and the rule-in cutoff troponin value or concentration at presentation is greater than 50 ng/L. In one example, the rule-out cutoff troponin value or concentration at presentation may be less than 6 ng/L, less than 5 ng/L, less than 4 ng/L, less than 3 ng/L, or less than 2 ng/L. In another example, the rule-in cutoff troponin value or concentration at presentation

may be, for example, greater than 50 ng/L, greater than 60 ng/L, greater than 70 ng/L or greater than 80 ng/L.

As illustrated in Table 1, patients may also be classified as “indeterminate” because the troponin value or concentration measured at presentation is greater than the rule-out troponin value or concentration and less than the rule-in cutoff troponin value or concentration at presentation. For a patient classified as indeterminate, a clinician may find it useful to evaluate the change (also referred to as “delta”) in troponin concentration in samples taken from the subject over time, at any suitable time interval. Examples of time intervals between the collection of two samples include 30 minutes, 45 minutes, 60 minutes, 75 minutes, 90 minutes, 105 minutes, 120 minutes, 150 minutes, or 180 minutes. The change (delta) in troponin concentration between, for example, when the patient first arrives at the emergency department and the first sample is taken and when the second sample is taken, may be used to determine whether the patient is likely to be suffering from a health-relevant cardiac event. If the “indeterminate” (or “indeterminant”) patient exhibits increases in measured troponin concentrations over time, the patient is typically more likely to be suffering from a health-relevant cardiac event. In contrast, if the patient exhibits negligible change in measured troponin concentrations over time, the patient is less likely to be suffering from a health-relevant cardiac event. Exemplary deltas or changes of troponin concentration over time are shown in Table 2. The magnitude of the change (also referred to as “delta”) determines whether the patient is more likely to be suffering from a health-relevant cardiac event or not.

TABLE 2

| | Troponin value or concentration |
|---|---|
| Rule-out cutoff | <5 ng/L at presentation and/or |
| | <1 ng/L delta at 1 hour after presentation (<1 |
| | ng/L/h) |
| Indeterminate | Neither ruled-in nor ruled-out |
| Rule-in cutoff | >15 ng/L troponin at presentation and/or |
| | >15 ng/L delta at 1 hour after presentation (>15 |
| | ng/L/h) |

In the example set forth in Table 2, when the patient presented with a troponin value or concentration at presentation of less than 5 ng/L and/or a change (delta) of less than 1 ng/L/hour, the patient is considered unlikely to be suffering from a health-relevant cardiac event; when the patient presented with a troponin value or concentration of at least 15 ng/L and/or a change (delta) of greater than 15 ng/L/hour, the patient is considered likely to be suffering from MACE. In some examples, if the troponin value or concentration at presentation in a sample taken from a patient is less than 50 ng/L but greater than 4 ng/L, and the delta in the troponin value or concentration is, for example, less than 1 ng/L/hour, then the subject may be ruled out as experiencing cardiac injury and/or ruled-out as cardiac patient. Further, if at presentation a sample taken from a patient contains at least 15 ng/L troponin and the delta in the troponin value or concentration is, for example, greater than 15 ng/L/hour, greater than 20 ng/L/hour, greater than 50 ng/L/hour, or greater than 100 ng/L/hour, then the patient may be ruled in as experiencing a health-relevant cardiac event and/or ruled-in as cardiac patient. If the patient continues to be indeterminate after one hour, the clinician may wait for an appropriate additional period of time (for example, one, two, three, or more hours) and take another sample from the patient. At that point, the troponin value or concentration or the change (delta) may either rule the patient in or out, or the patient may continue to be indeterminate and, as a result, may be kept at the hospital for further evaluation.

A person having skill in the art will recognize that the troponin reference value (including a rule-in cutoff troponin value or a rule-out cutoff troponin value or both) may be chosen to optimize the negative predictive value and the positive predictive value for a specific population. For example, the rule-out cutoff value may be selected to optimize the negative predictive value (that is the number of patients with a negative result who do not have the disease). Thus, for example, the rule-in cutoff and rule-out cutoff may be (i) adjusted based on the criteria used to create the disease reference populations, (ii) optimized prior to clinical feedback based on disease reference patients and healthy reference patients, and/or (iii) adjusted based on clinical feedback showing that there are too many patients being included in the disease reference patients that, upon further evaluation by a clinician, are not diagnosed as cardiac patients (false positives). The rule-in cutoff and the rule-out cutoff may also be adjusted depending on clinical feedback showing that there are too many patients being included in the healthy reference patients that, upon further evaluation by a clinician, are diagnosed as cardiac patients (false negatives). In sum, the values provided in Tables 1 and 2, including the delta values in Table 2, are only exemplary and may be adjusted as necessary to maximize sensitivity (how well a test correctly identifies people who have the disease) and specificity (how well a test correctly identifies people who do not have the disease).

Subjects or patients that are found to have a concentration of troponin greater than the rule-in cutoff (either at presentation or determined using a change in troponin concentration over time) may be diagnosed as cardiac patients and/or as patients with cardiac injury. Patients that are found to have a concentration of troponin lower than the rule-out cutoff (either at presentation or determined using a change in troponin concentration over time) may be found unlikely to be exhibiting cardiac injury (also referred to as “non-cardiac patients”). But the present invention may be of particular advantage for patients that are found to be indeterminates (either at presentation or determined using a change in troponin concentration over time) because determining and/or assessing the risk of MACE in these patients, where the troponin level or value alone may not suffice to immediately and/or definitely rule in or rule out a patient as having a cardiac event, is particularly difficult.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

The present disclosure provides for the following numbered statements, the numbering of which is not to be construed as designating levels of importance:

1. A computer-implemented method of determining a risk of a major adverse cardiovascular event, MACE, in a patient, the method comprising: (a) receiving, with a computing device, subject value data for the patient, the subject value data including (i) at least one troponin value, (ii) at least one demographic value, and (iii) a value for prior history of renal disease; (b) evaluating, with the computing device, the received subject value data of the patient based on a reference dataset indicative of reference subject values associated with one or more reference patients; wherein the reference dataset is indicative of reference subject values including (i) at least one troponin value, (ii) at least one demographic value, and (iii) a value for prior history of cardiac disease; and (c) determining the risk of MACE based on the evaluation from step (b).

2. The method according to Statement 1, wherein each of the subject value data and the reference subject values further comprise (iv) at least one of an erythrocyte mean corpuscular hemoglobin value and an electrolyte value.

3. The method according to any one of the preceding statements, wherein each of the subject value data and the reference subject values further comprise (v) a value for prior history of renal disease.

4. The method according to any one of the preceding statements, wherein the reference dataset is indicative of one or more threshold values for one or more of the reference subject values.

5. The method according to any one of the preceding statements, further comprising: determining one or more reference patients having a prior history of renal disease based on determining an estimated Glomerular Filtration Rate value (eGFR) or creatinine value.

6. The method according to any one of the preceding statements, further comprising: processing historic patient data indicative of a medical disease history of the patient; and determining the patient having a prior history of renal disease based on determining an estimated Glomerular Filtration Rate value or creatinine value.

7. The method according to any one of Statements 5 and 6, wherein a threshold indicative of renal disease is a creatinine value above of about 1.3 mg/dL.

8. The method according to any one of Statements 5 to 7, wherein a threshold indicative of renal disease is an eGFR value of about 60 mL/min/1.73 m².

9. The method according to any one of the preceding statements, further comprising: processing historic patient data indicative of a medical disease history of the patient; and determining the patient having a prior history of cardiac disease based on determining whether the patient was previously diagnosed with a cardiac disease.

10. The method according to any one of the preceding statements, wherein each of the subject value data and the reference subject values further comprise an electrolyte value; and wherein the at least one electrolyte value of the subject value data includes at least one of a magnesium value and a potassium value.

11. The method according to any one of the preceding statements, wherein the at least one demographic value includes at least one of a gender value, a racial value, and an age value.

12. The method according to any one of the preceding statements, further comprising one or more of: determining a gender of the patient based on the demographic value; classifying the patient into an age group of a plurality of predefined age groups based on the demographic value; classifying the patient into a race group of a plurality of predefined race groups based on the demographic value; and selecting the reference dataset based on the at least one demographic value.

13. The method according to any one of the preceding statements, further comprising: receiving further subject value data for the patient, the further subject value data including one or more of a ratio of oxyhemoglobin to hemoglobin, a ratio of neutrophils per leukocytes, a ratio of nucleated erythrocytes to leukocyte, and a ratio of eosinophils to leukocytes; and evaluating the further subject value data based on one or more further reference subject values indicated by the reference dataset, the one or more reference subject values being indicative of one or more of a ratio of oxyhemoglobin to hemoglobin, a ratio of neutrophils per leukocytes, a ratio of nucleated erythrocytes to leukocyte, and a ratio of eosinophils to leukocytes.

14. A computer program, which, when executed by one or more processors of a computing device, instructs the computing device to perform steps of the method according to any one of the preceding statements.

15. A non-transitory computer-readable medium storing a computer program according to Statement 14.

16. A computing device configured to perform steps of the method according to any one of Statements 1 to 13.

17. A computer-implemented method of determining a risk of major adverse cardiovascular event, MACE, in a patient, the method comprising: (a) receiving, with a computing device, subject value data for the patient, the subject value data including (i) at least one troponin value, (ii) at least one demographic value, and (iii) at least one of an erythrocyte mean corpuscular hemoglobin value and an electrolyte value; (b) evaluating, with the computing device, the received subject value data of the patient based on a reference dataset indicative of reference subject values associated with one or more reference patients who had been previously assessed for MACE; wherein the reference dataset is indicative of reference subject values including (i) at least one troponin value, (ii) at least one demographic value, and (iii) a value for prior history of cardiac disease; and (c) determining the risk of MACE based on the evaluation from step (b).

18. The method according to Statement 17, wherein each of the subject value data and the reference subject values further comprise (v) a value for prior history of renal disease.

19. The method according to Statement 17, wherein each of the subject value data and the reference subject values further comprise (v) a value for prior history of cardiac disease.

20. The method according to Statement 17, wherein each of the subject value data and the reference subject values further comprise (v) a value for prior history of cardiac disease and a value for prior history of renal disease.

21. The method according to any one of Statements 17-20, wherein the reference dataset is indicative of one or more threshold values for one or more of the reference subject values.

22. The method according to any one of Statements 17-21, further comprising: determining one or more reference patients having a prior history of renal disease based on determining an estimated Glomerular Filtration Rate value (eGFR) or creatinine value.

23. The method according to any one of Statements 17-22, further comprising: processing historic patient data indicative of a medical disease history of the patient; and determining the patient having a prior history of renal disease based on determining an estimated Glomerular Filtration Rate value or creatinine value.

24. The method according to any one of Statements 22 and 23, wherein a threshold indicative of renal disease is a creatinine value above of about 1.3 mg/dL.

25. The method according to any one of Statements 22 to 24, wherein a threshold indicative of renal disease is an eGFR value of about 60 mL/min/1.73 m².

26. The method according to any one of Statements 17-25, further comprising: processing historic patient data indicative of a medical disease history of the patient; and determining the patient having a prior history of cardiac disease based on determining whether the patient was previously diagnosed with a cardiac disease.

27. The method according to any one of Statements 17-26, wherein each of the subject value data and the reference subject values further comprise an electrolyte value; and wherein the at least one electrolyte value of the subject value data includes at least one of a magnesium value and a potassium value.

28. The method according to any one of Statements 17-27, wherein the at least one demographic value includes at least one of a gender value, a racial value, and an age value.

29. The method according to any one of Statements 17-28, further comprising one or more of: determining a gender of the patient based on the demographic value; classifying the patient into an age group of a plurality of predefined age groups based on the demographic value; classifying the patient into a race group of a plurality of predefined race groups based on the demographic value; and selecting the reference dataset based on the at least one demographic value.

30. The method according to any one of Statements 17-29, further comprising: receiving further subject value data for the patient, the further subject value data including one or more of a ratio of oxyhemoglobin to hemoglobin, a ratio of neutrophils per leukocytes, a ratio of nucleated erythrocytes to leukocyte, and a ratio of eosinophils to leukocytes; and evaluating the further subject value data based on one or more further reference subject values indicated by the reference dataset, the one or more reference subject values being indicative of one or more of a ratio of oxyhemoglobin to hemoglobin, a ratio of neutrophils per leukocytes, a ratio of nucleated erythrocytes to leukocyte, and a ratio of eosinophils to leukocytes.

31. A computer program, which, when executed by one or more processors of a computing device, instructs the computing device to perform steps of the method according to any one of Statements 17-30.

32. A non-transitory computer-readable medium storing a computer program according to Statement 31.

33. A computing device configured to perform steps of the method according to any one of Statements 17 to 30.

1. A computer-implemented method of determining a risk of a major adverse cardiovascular event, MACE, in a patient, the method comprising:
(a) receiving, with a computing device, subject value data for the patient, the subject value data including (i) at least one troponin value, (ii) at least one demographic value, and (iii) a value for prior history of cardiac disease;
(b) evaluating, with the computing device, the received subject value data of the patient based on a reference dataset indicative of reference subject values associated with one or more reference patients;
wherein the reference dataset is indicative of reference subject values including (i) at least one troponin value, (ii) at least one demographic value, and (iii) a value for prior history of cardiac disease; and
(c) determining the risk of MACE based on the evaluation from step (b).

2. The method according to claim 1, wherein each of the subject value data and the reference subject values further comprise (iv) at least one of an erythrocyte mean corpuscular hemoglobin value and an electrolyte value.

3. The method according to claim 1, wherein each of the subject value data and the reference subject values further comprise (v) a value for prior history of renal disease.

4. The method according to claim 1,
wherein the reference dataset is indicative of one or more threshold values for one or more of the reference subject values.

5. The method according to claim 1, further comprising:
determining one or more reference patients having a prior history of renal disease based on determining an estimated Glomerular Filtration Rate value (eGFR) or creatinine value.

6. The method according to claim 1, further comprising:
processing historic patient data indicative of a medical disease history of the patient; and
determining the patient having a prior history of renal disease based on determining an estimated Glomerular Filtration Rate value (eGFR) or creatinine value.

7. The method according to claim 5, wherein a threshold indicative of renal disease is a creatinine value above of about 1.3 mg/dL.

8. The method according to claim 5, wherein a threshold indicative of renal disease is an eGFR value of about 60 mL/min/1.73 m².

9. The method according to claim 1, further comprising: processing historic patient data indicative of a medical disease history of the patient; and
determining the patient having a prior history of cardiac disease based on determining whether the patient was previously diagnosed with a cardiac disease.

10. The method according to claim 1,
wherein each of the subject value data and the reference subject values further comprise an electrolyte value; and
wherein the at least one electrolyte value of the subject value data includes at least one of a magnesium value and a potassium value.

11. The method according to claim 1,
wherein the at least one demographic value includes at least one of a gender value, a racial value, and an age value.

12. The method according to claim 1, further comprising one or more of:
determining a gender of the patient based on the demographic value;
classifying the patient into an age group of a plurality of predefined age groups based on the demographic value;
classifying the patient into a race group of a plurality of predefined race groups based on the demographic value; and
selecting the reference dataset based on the at least one demographic value.

13. The method according to claim 1, further comprising:
receiving further subject value data for the patient, the further subject value data including one or more of a ratio of oxyhemoglobin to hemoglobin, a ratio of neutrophils per leukocytes, a ratio of nucleated erythrocytes to leukocyte, and a ratio of eosinophils to leukocytes; and
evaluating the further subject value data based on one or more further reference subject values indicated by the reference dataset, the one or more reference subject values being indicative of one or more of a ratio of oxyhemoglobin to hemoglobin, a ratio of neutrophils per leukocytes, a ratio of nucleated erythrocytes to leukocyte, and a ratio of eosinophils to leukocytes.

14. A computer program, which, when executed by one or more processors of a computing device, instructs the computing device to perform steps of the method according to claim 1.

15. A non-transitory computer-readable medium storing a computer program according to claim 14.

16. A computing device configured to perform steps of the method according to claim 1.

17. The method according to claim 1, wherein each of the subject value data and the reference subject values further comprises:
at least one of an erythrocyte mean corpuscular hemoglobin value and an electrolyte value; and
a value for prior history of renal disease.

18. The method according to claim 6, wherein a threshold indicative of renal disease is a creatinine value above of about 1.3 mg/dL.

19. The method according to claim 6, wherein a threshold indicative of renal disease is an eGFR value of about 60 mL/min/1.73 m².
